(19) United States
(12) Patent Application Publication
(10) Pub. No.: US 2014/0121489 A1
(43) Pub. Date: May 1, 2014

(54) MEDICAL IMAGING SYSTEM AND A PORTABLE MEDICAL IMAGING DEVICE FOR PERFORMING IMAGING

(71) Applicant: General Electric Company, Schenectady, NY (US)

(72) Inventor: Mohan Krishna KOMMU CHS, Bangalore (IN)

(73) Assignee: General Electric Company, Schenectady, NY (US)

(21) Appl. No.: 14/067,194

(22) Filed: Oct. 30, 2013

(30) Foreign Application Priority Data
Oct. 31, 2012 (IN) 4538/CHE/2012

(51) Int. Cl. A61B 8/00; A61B 6/00

(52) U.S. Cl. CPC A61B 8/4427; A61B 6/4405; A61B 8/461; A61B 6/461
USPC 600/407

(57) ABSTRACT

A portable medical imaging device for performing imaging procedures. The portable medical imaging device comprises a processor configured to analyze location information associated with the portable medical imaging device and determine whether the location information is linked to a location of the performing an imaging procedure. The processor selects an imaging configuration from a plurality of imaging configurations associated with an imaging procedure to be performed in the location, wherein each imaging configuration of the plurality of imaging configurations is associated with a type of imaging procedure. The processor is further configured to load the imaging configuration to perform the imaging procedure. The portable medical device (200) for performing medical procedures further comprises at least one memory communicably coupled to the processor for storing the plurality of imaging configurations.

### TECHNICAL FIELD

Embodiments of the present invention relate to portable medical imaging devices, and, more specifically, to a portable medical imaging devices that can automatically reconfigure for performing imaging procedures in different locations.

### BACKGROUND OF THE INVENTION

Medical imaging systems such as ultrasound imaging systems are used in different applications to image different regions or areas (e.g. different organs) of patients or other objects. For example, an ultrasound imaging system may be utilized to generate an image of organs, vasculature, heart, or other portions of the body. Ultrasound imaging systems are generally located at a medical facility, for example, a hospital or an imaging center. However, not all people have access to a medical facility. In particular, individuals at nursing homes, under home care, or in rural areas may not be capable of attending a medical facility for ultrasound imaging.

Portable medical imaging devices may be utilized to acquire images of a patient at locations remote from a medical facility. For example, a portable ultrasound imaging system acquires imaging data at the remote location that then may be provided to the medical facility. Thereafter, the imaging data is used to generate an image at the medical facility. In particular, the imaging data is only capable of being extracted from the portable ultrasound imaging system and sent to the medical facility. Extracting the imaging data is commonly performed by downloading the imaging data onto a data disc or other media that is mailed to the medical facility. These portable medical imaging devices may be moved from one location to another for different types of imaging procedures such as cardiac imaging and an obstetric imaging. A user of the portable medical imaging device needs to manually set an imaging configuration associated with these imaging procedures as they vary based on a type of the imaging procedure. This consumes a lot of time in preparing the portable medical imaging device for the imaging procedure when moved from one location to another. Thus there is a need for a portable medical imaging device that can reduce the time in configuring itself for performing different imaging procedures at multiple locations.

### BRIEF DESCRIPTION OF THE INVENTION

The above-mentioned shortcomings, disadvantages and problems are addressed herein which will be understood by reading and understanding the following specification.

According to an embodiment of the present invention, there is provided a portable medical imaging device for performing imaging procedures. The portable medical imaging device comprises a processor configured to analyze location information associated with the portable medical imaging device. Then it is determined whether the location information is linked to a location of the performing an imaging procedure. The processor selects an imaging configuration from a plurality of imaging configurations associated with an imaging procedure to be performed in the location each imaging configuration of the plurality of imaging configurations is associated with a type of imaging procedure. The plurality of imaging procedures is stored in at least one memory communicably coupled to the processor. The selected imaging configuration is loaded to perform the imaging procedure.

In an embodiment, a medical imaging system for performing imaging procedures is provided. The medical imaging system includes a portable medical imaging device having a processor configured to analyze location information associated with the portable medical imaging device. Then it is determined whether the location information is linked to a location of the performing an imaging procedure. The processor selects an imaging configuration from a plurality of imaging configurations associated with an imaging procedure to be performed in the location each imaging configuration of the plurality of imaging configurations is associated with a type of imaging procedure. The plurality of imaging procedures is stored in at least one memory communicably coupled to the processor. The selected imaging configuration is loaded to perform the imaging procedure.

In an embodiment, a method of automatically configuring a portable medical imaging device for performing imaging is provided. The method comprises: detecting a presence of the portable medical imaging device in a location for performing an imaging procedure, wherein the presence is detected based on location information associated with the portable medical imaging device; selecting an imaging configuration from a plurality of imaging configurations associated with an imaging procedure to be performed in the location, each imaging configuration of the plurality of imaging configurations is associated with a type of imaging procedure; and loading the imaging configuration in the portable medical imaging device to perform the imaging procedure.

### BRIEF DESCRIPTION OF THE DRAWINGS

FIG. 1 is a schematic illustration of a portable medical imaging device such as an ultrasound imaging system;

FIG. 2 is a schematic illustration of a portable ultrasound imaging system for performing imaging procedures in accordance with an embodiment of the invention;

FIG. 3 is a schematic illustration of a medical imaging system wherein a portable ultrasound imaging system including a position detection circuitry that communicates with a GPS server in accordance with an embodiment of the invention;

FIG. 4 is a schematic illustration of a medical imaging system wherein a portable ultrasound imaging system including a position detection circuitry that communicates with at least one wireless detector in accordance with an embodiment of the invention;

FIG. 5 illustrates a method for automatically configuring a portable medical imaging device for performing imaging in accordance with an embodiment of the invention;

FIG. 6 illustrates a method for automatically configuring the portable medical imaging device for performing imaging in accordance with an embodiment of the invention; and

FIG. 7 illustrates a method for automatically configuring the portable medical imaging device for performing imaging in accordance with an embodiment of the invention.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

To the extent that the figures illustrate diagrams of the functional blocks of various embodiments, the functional blocks are not necessarily indicative of the division between hardware circuitry. One or more of the functional blocks (e.g., processors or memories) may be implemented in a single piece of hardware (e.g., a general purpose signal processor or random access memory, hard disk, or the like) or multiple pieces of hardware. Similarly, the programs may be standalone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

A portable medical imaging device for performing imaging procedures is disclosed. The portable medical imaging device comprises a processor configured to analyze location information associated with the portable medical imaging device. Then it is determined whether the location information is linked to a location of the performing an imaging procedure. The processor selects an imaging configuration from a plurality of imaging configurations associated with an imaging procedure to be performed in the location each imaging configuration of the plurality of imaging configurations is associated with a type of imaging procedure. The plurality of imaging procedures is stored in at least one memory communicably coupled to the processor. The selected imaging configuration is loaded to perform the imaging procedure.

Although the various embodiments are described with respect to an ultrasound imaging system, the various embodiments may be utilized with any suitable imaging systems, for example, X-ray or the like.

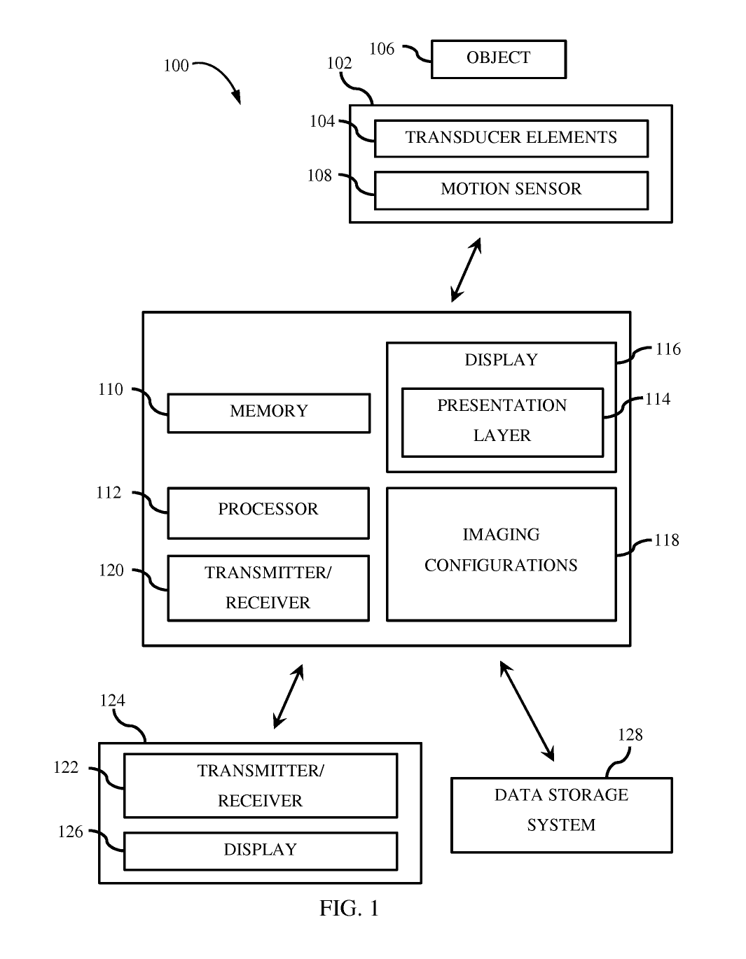

FIG. 1 is a schematic illustration of a portable medical imaging device such as an ultrasound imaging system 100. The ultrasound imaging system 100 may be a portable or a handheld ultrasound imaging system. For example, the ultrasound imaging system 100 may be similar in size to a smartphone, a personal digital assistant or a tablet. In some embodiments, the ultrasound imaging system 100 may be configured as a laptop or a cart based system. The ultrasound imaging system 100 may be transportable to a remote location, such as a nursing home, a medical facility, rural area, or the like. The ultrasound imaging system 100 may be moved from one imaging room to another in a particular location such as a medical facility. These imaging rooms may include but are not limited to a cardiac imaging room, an obstetric imaging room, and an emergency room.

A probe 102 is in communication with the ultrasound imaging system 100. The probe 102 may be mechanically coupled to the ultrasound imaging system 100. Alternatively, the probe 102 may wirelessly communicate with the ultrasound imaging system 100. The probe 102 includes transducer elements 104 that emit ultrasound pulses to an object 106 to be scanned, for example an organ of a patient. The ultrasound pulses may be back-scattered from structures within the object 106, such as blood cells or muscular tissue, to produce echoes that return to the transducer elements 104. The transducer elements 104 generate ultrasound image data based on the received echoes. The probe 102 also includes a motion sensor 108 in accordance with an embodiment. The motion sensor 108 may include but is not limited to, an accelerometer, a magnetic sensor and a gyro sensor. The motion sensor 108 is configured to identify the position and orientation of the probe 102 on the object 106. The position and orientation may be identified in real-time, when a medical expert is manipulating the probe 102. The term “real-time” includes an operation or procedure that is performed without any intentional delay. The probe 102 transmits the ultrasound image data to the ultrasound imaging system 100. The ultrasound imaging system 100 includes a memory 110 that stores the ultrasound image data. The memory 110 may be a database, random access memory, or the like. In an embodiment, the memory 110 is a secure encrypted memory that requires a password or other credentials to access the image data stored therein. The memory 110 may have multiple levels of security. For example, a surgeon or doctor may have access to all of the data stored in the memory 110, whereas, a technician may have limited access to the data stored in the memory 110. In an embodiment, a patient may have access to the ultrasound image data related to the patient, but is restricted from all other data. A processor 112 accesses the ultrasound image data from the memory 110. The processor 112 may be a logic based device, such as one or more computer processors or microprocessors. The processor 112 generates an image based on the ultrasound image data. The image is displayed on a presentation layer 114, which may be, for example, a graphical user interface (GUI) or other displayed user interface, such as a virtual desktop. The presentation layer 114 may be a software based display that is accessible from multiple locations.

The presentation layer 114 displays the image on a display 116 provided within the ultrasound imaging system 100. The display 116 may be a touch sensitive screen. Alternatively, the presentation layer 114 may be accessible through a web-based browser, local area network, or the like. In such an embodiment, the presentation layer 114 may be accessible remotely as a virtual desktop that displays the presentation layer 114 in the same manner as the presentation layer 114 is displayed in the display 116.

The ultrasound imaging system 100 includes imaging configurations 118 associated with different imaging procedures that can be performed. The imaging procedures include for example, obstetric imaging, cardiac imaging and abdominal imaging. Based on an imaging procedure to be performed a corresponding imaging configuration needs to be set. The imaging configuration may be set by a user in the ultrasound imaging system 100. The imaging configurations may be pre-stored in the ultrasound imaging system 100. The imaging configuration may include various parameters such as frequency, a speckle reduction imaging, time gain compensation, scan depth, scan format, image frame rate, field of view, focal point, scan lines per image frame, number of ultrasound beams and pitch of the transducer elements. These parameters vary for different imaging configurations. For example, the ultrasound imaging system 100 may be used for cardiac application by configuring a cardiac imaging configuration. Thereafter an abdominal imaging configuration stored in the ultrasound imaging system 100 needs to be set for performing the abdominal imaging application. For the cardiac application, an image frame rate is an important factor. Therefore, the ultrasound imaging system 100 is set to switch off few imaging filters such as a frame averaging filter and a speckle reduction imaging filter, and also vary some parameters like narrow field of view, single focal point, lesser number of scan lines per image frame. In an abdominal application, resolution may be an important parameter. Thus the ultrasound imaging system 100 turns on medium or high frame averaging filter and a speckle reduction imaging filter. Further, some parameters may be also set for example multiple focal points, wide field of view, more number of scan lines per image frame (i.e. higher line density), and transmission of multiple ultrasound beams.

The ultrasound imaging system 100 also includes a transmitter/receiver 120 that communicates with a transmitter/receiver 122 of a workstation 124. For example, the workstation 124 may be positioned at a location, such as a hospital, imaging center, or other medical facility. The workstation 124 may be a computer, tablet-type device, or the like. The workstation 124 may be any type of computer or end user device. The workstation 124 includes a display 126. The workstation 124 communicates with the ultrasound imaging system 100 to display an image based on image data acquired by the ultrasound imaging system 100 on the display 126. The workstation 124 also includes any suitable components image viewing, manipulation, etc.

The ultrasound imaging system 100 and the workstation 124 communicate through the transmitter/receivers 120 and 122, respectively. The ultrasound imaging system 100 and the workstation 124 may communicate over a local area network. For example, the ultrasound imaging system 100 and the workstation 124 may be positioned in separate remote locations of a medical facility and communicate over a network provided at the facility. In an exemplary embodiment, the ultrasound imaging system 100 and the workstation 124 communicate over an internet connection, such as through a web-based browser.

An operator may remotely access imaging data stored on the ultrasound imaging system 100 from the workstation 124. For example, the operator may log onto a virtual desktop or the like provided on the display 126 of the workstation 124. The virtual desktop remotely links to the presentation layer 114 of the ultrasound imaging system 100 to access the memory 110 of the ultrasound imaging system 100. The memory 110 may be secured and encrypted to limit access to the image data stored therein. The operator may input a password to gain access to at least some of the image data.

Once access to the memory 110 is obtained, the operator may select image data to view. It should be noted that the image data is not transferred to the workstation 124. Rather, the image data is processed by the processor 112 to generate an image on the presentation layer 114. For example, the processor 112 may generate a DICOM image on the presentation layer 114. The ultrasound imaging system 100 transmits the presentation layer 114 to the display 126 of the workstation 124 so that the presentation layer 114 is viewable on the display 126. In an embodiment, the workstation 124 may be used to manipulate the image on the presentation layer 114. The workstation 124 may be used to change an appearance of the image, such as rotate the image, enlarge the image, adjust the contrast of the image, or the like. Moreover, an image report may be input at the workstation 124. For example, an operator may input notes, analysis, and/or comments related to the image. In an embodiment, the operator may input landmarks or other notations on the image. The image report is then saved to the memory 110 of the ultrasound imaging system 100. Accordingly, the operator can access images remotely and provide analysis of the images without transferring the image data from the ultrasound imaging system 100. The image data remains stored only on the ultrasound imaging system 100 so that the data remains restricted only to individuals with proper certification.

In an embodiment, the ultrasound imaging system 100 is capable of simultaneous scanning and image data acquisition. The ultrasound imaging system 100 may be utilized to acquire a first set of imaging data, while a second set of imaging data is accessed to display on the display 126 of the workstation 124 an image based on the second set of imaging data. The ultrasound imaging system 100 may also capable of transferring the image data to a data storage system 128 present in a remote location. The ultrasound imaging system 100 communicates with the data storage system 128 over a wired or wireless network.

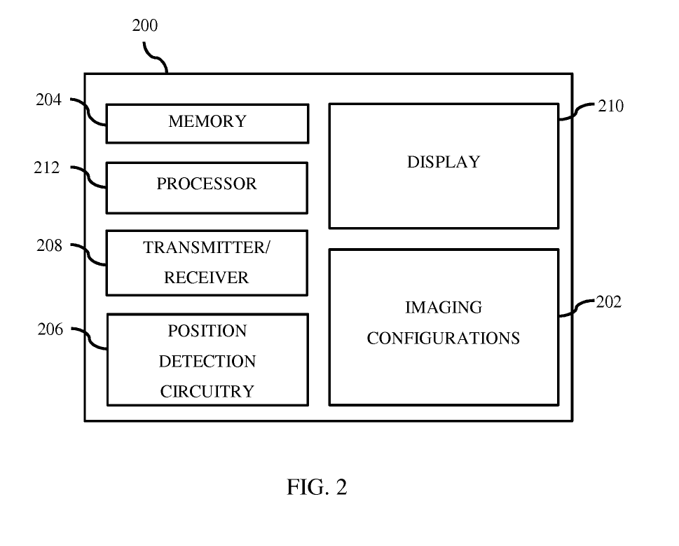

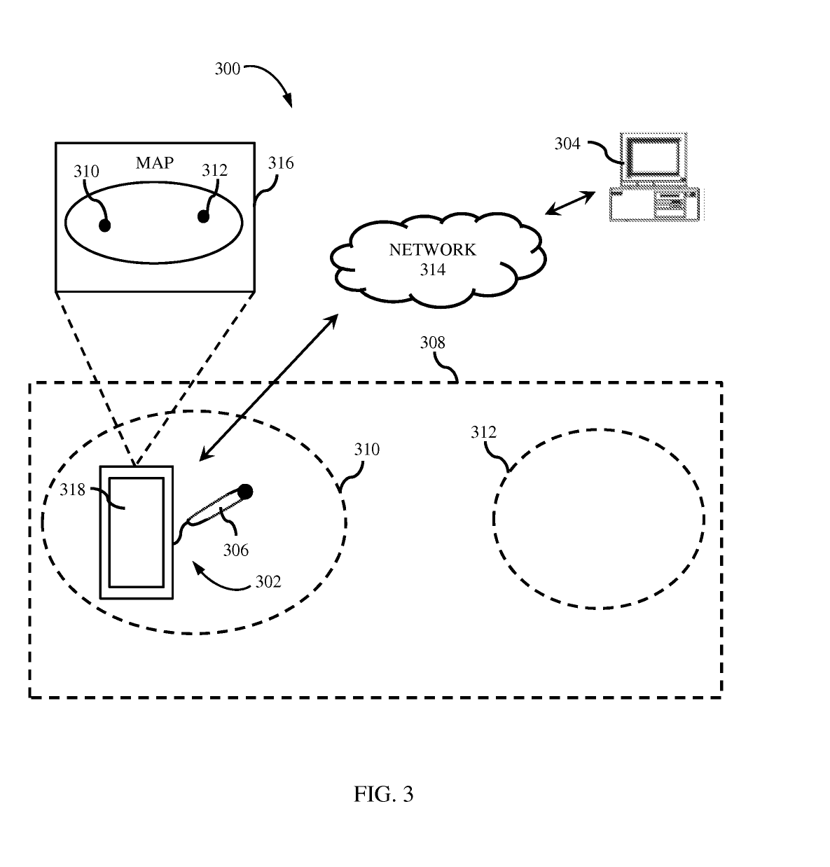

FIG. 2 is a schematic illustration of a portable ultrasound imaging system 200 for performing imaging procedures in accordance with an embodiment. The portable ultrasound imaging system 200 may be moved from one location to another by a user. The user may be for example a clinician, a doctor, and a medical expert. The location may be an obstetric imaging room, a cardiac imaging room, an abdominal imaging room, and an emergency room. For example the portable ultrasound imaging system 200 may be moved from an obstetric imaging room to a cardiac imaging room for performing a cardiac imaging. To conduct the cardiac imaging corresponding imaging configuration (i.e. a cardiac imaging configuration) need to be set in the portable ultrasound imaging system 200. The cardiac imaging configuration is selected from the imaging configurations 202 stored in a memory 204. The portable ultrasound imaging system 200 includes a position detection circuitry 206 for determining its position. Thus the position detection circuitry 206 identifies location information of the portable ultrasound imaging system 200 that moves from one location to another location. The location information may include location coordinates associated with the position of the portable ultrasound imaging system 200. The location coordinates may be determined using a Global Positioning System (GPS) technique. Here the position detection circuitry 206 may detect the location coordinates and present them in a map. The map may show different locations where the portable ultrasound imaging system 200 moves. The portable ultrasound imaging system 200 includes a transmitter/receiver 208 that communicates with a GPS system to receive a corresponding map that shows the locations traveled. In an embodiment one or more maps may be pre-stored in the memory 204. The map along with the location information may be presented or displayed in a display 210 to the user. This is further explained in detail in conjunction with FIG. 3.

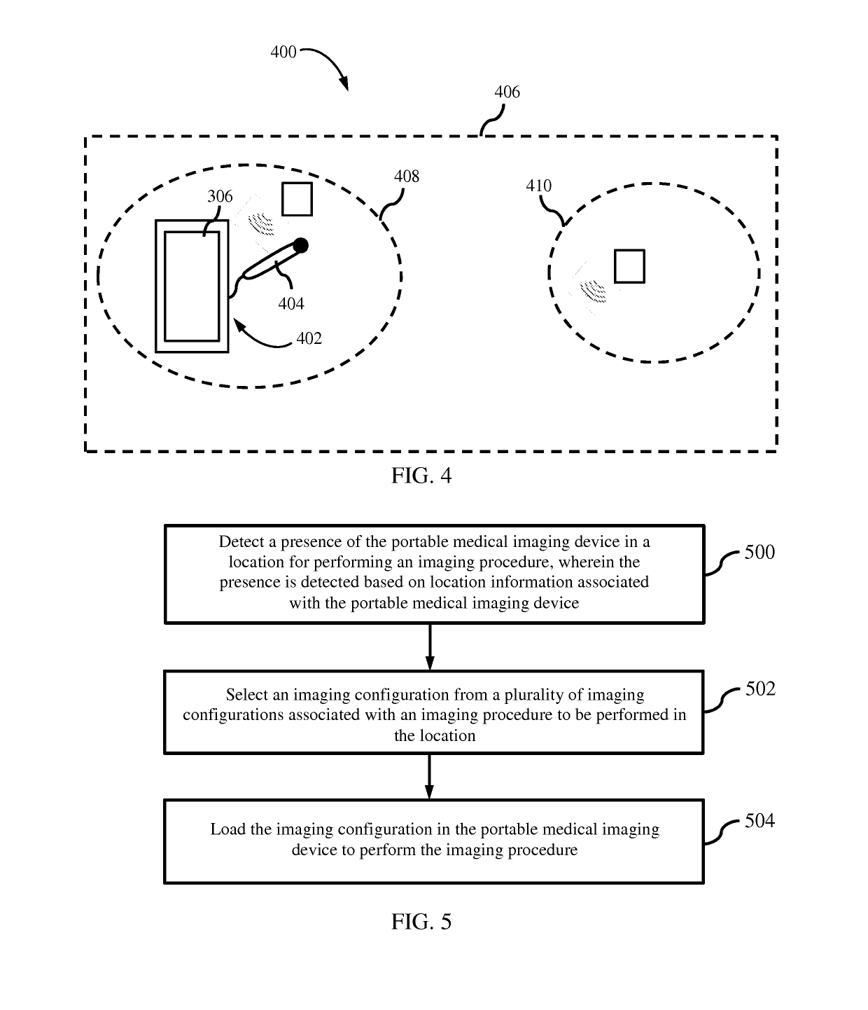

In an embodiment, the position detection circuitry 206 may operate based on a near field communication technique and/or Bluetooth® technique to determine the location information. In this case the position detection circuitry 206 may communicate with wireless detectors present in various locations to determine the location information of the portable ultrasound imaging system 200. The position detector circuitry 206 may communicate with the wireless detectors using the transmitter/receiver 208. Further it may be contemplated that the position detection circuitry 206 may use any other wireless techniques to determine the location information of the portable ultrasound imaging system 200. This is explained in detail in conjunction with FIG. 4. The location may be continuously determined when the portable ultrasound imaging system 200 is in transit from one location to another location. In an embodiment during transit a processor 208 may configure the portable ultrasound imaging system 200 in a standby mode. This is to save power consumption because the portable ultrasound imaging system 200 is not performing any imaging procedure. In the standby mode the position detection circuitry 206 may consume less power for its operation. In an embodiment, the position detection circuitry 206 may use a battery or a power source for supplying power for its operation. The position detection circuitry 206 communicates the location information to a processor 212. The processor 212 analyzes the location information and determines whether the location information is associated with a location of performing an imaging procedure or a location where the portable ultrasound imaging system 200 is destined to reach. If the location information is matching with the location then the processor 212 automatically selects an imaging configuration associated with the imaging procedure to be performed in the location. The imaging configuration is loaded or configured so that all parameters are set for performing the imaging procedure. As the imaging configuration is automatically selected and configured in the portable ultrasound imaging system 200 manual interventions of the user is eliminated. Moreover the imaging configuration is loaded based on the location information thus a delay in preparing the portable ultrasound imaging system 200 for performing the imaging procedure is also avoided.

Now explaining by way of an example, the portable ultrasound imaging system 200 may be moved by the user from an obstetric imaging room to a cardiac imaging room, location information of the portable ultrasound imaging system 200 may be determined on its transit. Once the portable ultrasound imaging system 200 completes obstetric imaging and moves out of the obstetric imaging room, the portable ultrasound imaging system 200 configures in a standby mode. Upon reaching the cardiac imaging room or reaching at the proximity of the cardiac imaging room, the portable ultrasound imaging system 200 automatically selects and loads a cardiac imaging configuration. Thus the portable ultrasound imaging system 200 is ready for performing the cardiac imaging procedure without any delay and manual intervention for loading the cardiac imaging procedure is avoided.

FIG. 3 illustrates a medical imaging system 300 wherein a portable ultrasound imaging system 302 including the position detection circuitry 206 (not shown in FIG. 3) communicates with a GPS server 304 in accordance with an embodiment. The portable ultrasound imaging system 302 includes a probe 306 that may be used on an object's body for performing an imaging procedure. As illustrated in FIG. 3, the probe 306 may have a wired connection with the portable ultrasound imaging system 302. However, it may be contemplated that the probe 306 may be wirelessly connected to the portable ultrasound imaging system 302. The portable ultrasound imaging system 302 is located in an area 308 such as, a hospital and a medical center. The area 308 includes a location 310 (for example a cardiac imaging room) and a location 312 (for example an obstetric imaging room). The area 308 is considered as a hospital or a medical center in FIG. 3 for sake of convenience of description however it may be envisioned that the area may be a much larger location such as, a state or country. In the location 310 the user may use the portable ultrasound imaging system 302 for performing a cardiac imaging procedure. The position detection circuitry 206 may have GPS communication capability and hence may use this technique for locating a position of the portable ultrasound imaging system 302 in the area 308. Initially the position detection circuitry 206 communicates with the GPS server 304 over a network 314 and receives a map 316 of the area 308. The network 314 may include but are not limited to, a third (3^{rd}) Generation communication (3G) network, a fourth (4^{th}) Generation communication (4G) network, and a Long Term Evolution communication (4G LTE) network. The map 316 is loaded into the portable ultrasound imaging system 302. The map 316 may be stored in a memory of the portable ultrasound imaging system 302. The position detection circuitry 206 locates coordinate positions associated with the portable ultrasound imaging system 302. The coordinate position may include latitude and longitude position information. The location 310 (i.e. the cardiac imaging room) may be the location that is identified, and the coordinate positions of the location 310 are also determined. The position detection circuitry 206 displays the position of the portable ultrasound imaging system 302 in the map 316. The map 316 may be presented in a display 318 of the portable ultrasound imaging system 302. The map 316 may also present the coordinate positions of the location 310 and indicate that it is the cardiac imaging room.

The user may decide to move the portable ultrasound imaging system 302 to the location 312 for an obstetric imaging procedure. In transit or when the cardiac imaging procedure is complete, the portable ultrasound imaging system 302 configures into a standby mode for consuming less power for its operation. In the standby mode the position detection circuitry 206 may operate using a battery to detect the location information of the portable ultrasound imaging system 302. In an embodiment the position detection circuitry 206 may continuously monitor location information of the portable ultrasound imaging system 302 in transit. Consequently, the map 316 presents the location information. In an embodiment the position detection circuitry 206 may monitor or retrieve the location information at predefined time intervals. Thus the map 316 may be updated with the location information at the predefined time intervals. In an embodiment, the map 316 may also present a path followed by the portable ultrasound imaging system 302 to reach the location 312 from the location 310. Moreover the map 316 may also be configured to present a distance between the location 310 and the location 312. When the portable ultrasound imaging system 302 transits the map 316 may be updated with distance information (i.e. distance left to be covered) until it reaches the location 312.

When the portable ultrasound imaging system 302 reaches the location 312 or is proximate to the location 312, the position detection circuit 206 activates the portable ultrasound imaging system 302. The map 316 indicates that the portable ultrasound imaging system 302 reached the location 312. In a wakeup mode an obstetric imaging configuration is identified from different imaging configurations present. The obstetric imaging configuration is identified in response to determining that the location information is associated with the obstetric imaging room. More specifically, in an embodiment a mapping between a location information, such as the location information of the location 312 and corresponding imaging configuration (i.e. the obstetric imaging configuration), may be stored in the portable ultrasound imaging system 302. This mapping may be stored in the form of a mapping table. Thus when the location information is determined the obstetric imaging configuration is identified from the mapping table. The obstetric imaging configuration is loaded or configured in the portable ultrasound imaging system 302 in order to prepare for the obstetric imaging procedure. As the portable ultrasound imaging system 302 is prepared when it moves into the location 312 the obstetric imaging procedure can be performed without long delays. This also eliminates the need of manual intervention from the user for preparing the portable ultrasound imaging system 302.

Now if the portable ultrasound imaging system 302 is carried out from the area 308 by the user, then in order to present the location information, the portable ultrasound imaging system 302 needs to retrieve a map associated with a new area where it is currently located. This map may be send by the GPS server 304 upon receiving a request.

Turning now to FIG. 4, the figure is a schematic illustration of a medical imaging system 400 wherein a portable ultrasound imaging system 402 including the position detection circuitry 206 (not shown in FIG. 4) communicates with at least one wireless detector in accordance with an embodiment. The portable ultrasound imaging system 402 includes a probe 404 that may be used on an object's body for performing an imaging procedure. As illustrated in FIG. 4, the probe 402 may have a wired connection with the portable ultrasound imaging system 402. However it may be contemplated that the probe 402 may be wirelessly connected to the portable ultrasound imaging system 402. The portable ultrasound imaging system 402 is located in an area 406 such as, a hospital and a medical center. The area 406 includes a location 408 (for example a cardiac imaging room) and a location 410 (for example an emergency room). The position detection circuitry 206 communicates with at least one wireless detector positioned in the area 406 for determining location information of the portable ultrasound imaging system 402. For instance the location 408 includes at least one wireless detector such as a wireless detector 412, and the location 410 includes at least one wireless detector such as a wireless detector 414. In an embodiment the wireless detector 412 and the wireless detector 414 may be positioned at an opening or door or entrance of the location 408 and the location 410 respectively. When the portable ultrasound imaging system 402 is positioned in the location 408 it communicates with the wireless detector 412 to detect the location information. The wireless detector 412 may be a Near Field Communication (NFC) based wireless detector and a Bluetooth® based wireless detector. Thus the wireless detector 412 detects whether any portable ultrasound imaging systems or any other wireless devices are present at a proximal distance to the location 408. So when the wireless detector 412 identifies that the portable ultrasound imaging system 402 is in the location 408 then the position detection circuitry 206 activates the portable ultrasound imaging system 402. Thereafter in an embodiment the wireless detector 412 communicates an identifier associated with the location 408 to the position detection circuitry 206. The identifier may indicate a type of imaging procedure (i.e. cardiac imaging procedure) to be performed in the location 408. The portable ultrasound imaging system 402 selects the cardiac imaging procedure from the pre-stored imaging configurations. In an embodiment a mapping table including a relationship between the identifier and corresponding imaging procedure may be stored in the portable ultrasound imaging system 402. The mapping table also includes other identifiers associated with various other locations (e.g. medical imaging rooms) in the area 406. The cardiac imaging procedure is loaded into the portable ultrasound imaging system 402 to perform the cardiac imaging procedure on an object.

The portable ultrasound imaging system 402 may be transported to the location 410 by the user for performing imaging procedures in the emergency room. During transportation the portable ultrasound imaging system 402 may be shifted to the standby mode. When the portable ultrasound imaging system 402 arrives proximal or within a detection range of the wireless detector 414 present at the entrance of the location 410, presence of the portable ultrasound imaging system 402 in the location 410 is detected. At this stage the position detection circuitry 206 may instruct the position detection circuitry 206 to shift to the wakeup mode. The detection range may be a range within which a wireless device can be detected by the wireless detector 414 using a NFC technique or a Bluetooth® technique. The wireless detector 414 also sends an identifier associated with the location 410 to the portable ultrasound imaging system 402. The identifier is used to determine the imaging procedures to be performed. The imaging configurations may be selected to be loaded based on user input based on requirements in the location 410. For instance in the emergency room different imaging procedures may need to be performed based on emergency requirements on the object i.e. a subject. In another scenario the imaging configurations may be loaded in the portable ultrasound imaging system 402. Thus by the time the portable ultrasound imaging system 402 enters the emergency room (i.e. reaches the location 410) all the necessary imaging configurations may be already loaded in the portable ultrasound imaging system 402. This avoids the delay in preparing the portable ultrasound imaging system 402 for performing the requirements in the emergency room. Moreover manual loading of the imaging configurations in the emergency room is also eliminated.

The medical imaging system 300 and the medical imaging system 400 operating using different techniques such as a GPS technique and a NFC and/or Bluetooth® technique are explained as different embodiments. However, it may be envisioned that a medical imaging system may include a portable ultrasound imaging system that may be configured to determine the location information during transit using both these techniques.

FIG. 5 illustrates a method for automatically configuring a portable medical imaging device for performing imaging in accordance with an embodiment. The portable medical imaging device such as a portable ultrasound imaging system may be similar in size to a smartphone, a personal digital assistant or a tablet. In some embodiments, the portable ultrasound imaging system may be configured as a laptop or a cart based system. The portable ultrasound imaging system may be transportable to a remote location, such as a nursing home, a medical facility, rural area, or the like. Further the portable ultrasound imaging system may be moved from one imaging room to another in a particular location such as a medical facility.

At block 500 presence of the portable medical imaging device in a location such as an imaging room is detected. The portable medical imaging device may need to perform an imaging procedure in the location. The presence in detected based on a location information of the portable medical imaging device. The location information may include location coordinates. Thereafter an imaging configuration associated with an imaging procedure to be performed in the location is selected from multiple imaging configurations at block 502. The location information is analyzed and it is determined whether the location information is associated with a location of performing the imaging procedure or a location where the portable medical imaging device is destined to reach. If the location information is matching with the location then an imaging configuration associated with the imaging procedure is automatically selected. Then at block 504 the imaging configuration is loaded or configured so that all parameters are set for performing the imaging procedure. The imaging configuration may include various parameters such as frequency, a speckle reduction imaging, time gain compensation, scan depth, scan format, image frame rate, field of view, focal point, scan lines per image frame, number of ultrasound beams and pitch of the transducer elements. These parameters vary for different imaging configurations. As the imaging configuration is automatically selected and configured in the portable medical imaging device manual interventions of the user is eliminated.

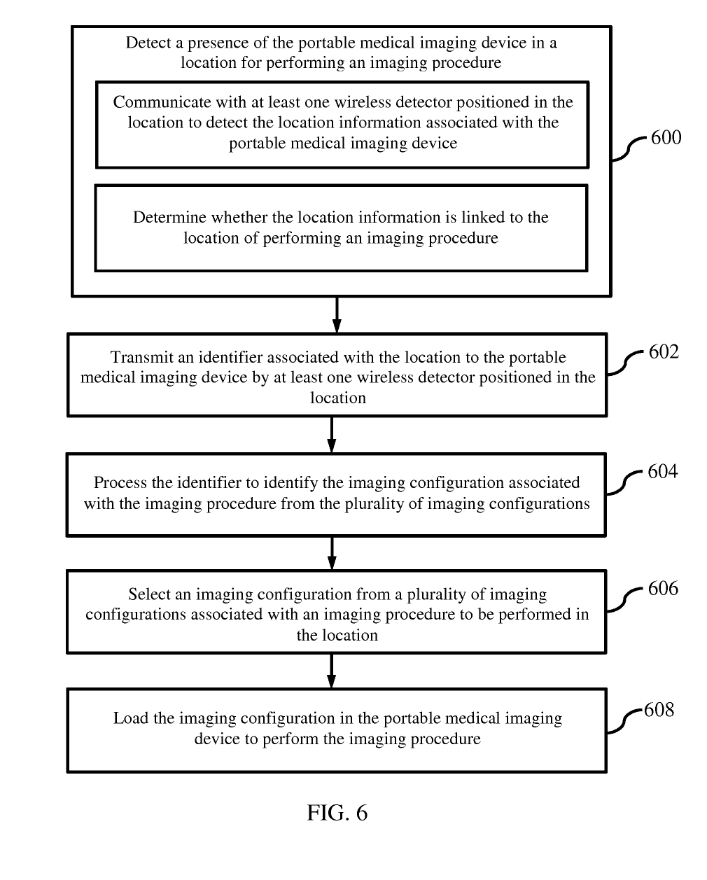

FIG. 6 illustrates a method for automatically configuring the portable medical imaging device for performing imaging in accordance with another embodiment. At block 600 the presence of the portable medical imaging device in a location such as an imaging room is detected. In order to detect the presence, one or more wireless detectors may be positioned in the location. The portable medical imaging device communicates with the one or more wireless detectors to detect its location information. The one or more wireless detectors may operate based on NFC technique and/or Bluetooth® technique. When the portable medical imaging device is at a proximal distance to the one or more wireless devices the communication is established. The proximal distance may be a NFC range or a Bluetooth range for communication. The location information of the portable medical imaging device is then used to determine whether this is linked to the location of performing the imaging procedure. If the location information is associated with the location, at block 602 the one or more wireless detectors transmit an identifier associated with the location to the portable medical imaging device. The identifier may indicate a type of imaging procedure (for example a cardiac imaging procedure) to be performed in the location. The identifier is processed to identify the imaging configuration from the multiple imaging configurations at block 604. Then at block 606, a corresponding imaging configuration associated with the imaging procedure is selected from the multiple imaging configurations to be performed in the location. In an embodiment, a mapping table including a relationship between the identifier and corresponding imaging procedure may be stored in the portable medical imaging device. The mapping table also includes other identifiers associated with various other locations (e.g. imaging rooms). The selected imaging procedure is loaded into the portable medical imaging device to perform the imaging procedure on the object at block 608.

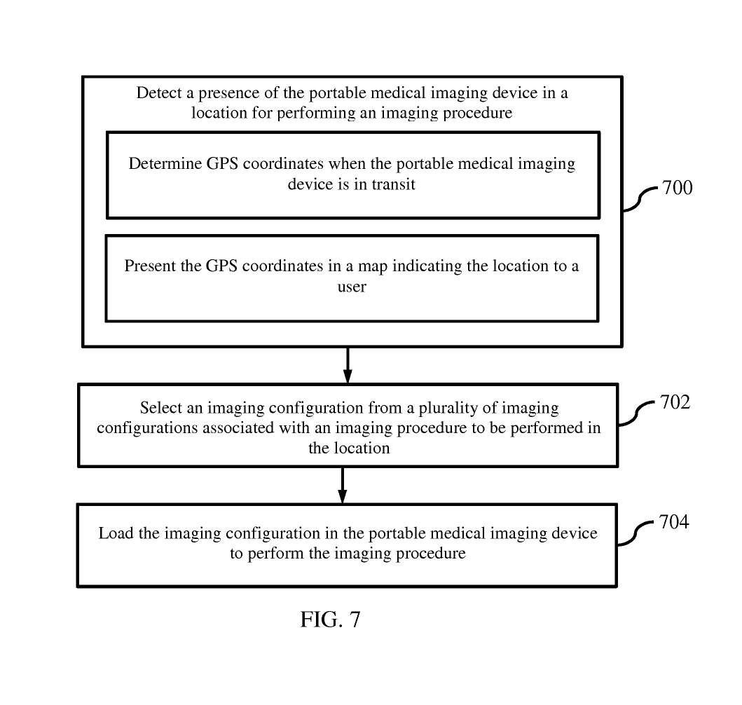

FIG. 7 illustrates a method for automatically configuring the portable medical imaging device for performing imaging in accordance with another embodiment. At block 700 the presence of the portable medical imaging device in a location such as an imaging room is detected. The presence is detected by determining location information associated with a position of the portable medical imaging device. The location information includes GPS coordinates associated with the position. The portable medical imaging device has GPS capability of identifying the position. Now if the GPS coordinates are associated with the location (i.e. the imaging room) then the presence in the location is confirmed. The GPS coordinates are displayed in a map through the portable medical imaging device. The map displays the location and presents the position along with the GPS coordinates to the user. The map may be displayed through a display of the portable medical imaging device.

Thereafter an imaging configuration associated with an imaging procedure to be performed in the location is selected from multiple imaging configurations at block 502. The location information is analyzed and it is determined whether the location information is associated with a location of performing the imaging procedure or a location where the portable medical imaging device is destined to reach. If the location information is matching with the location then an imaging configuration associated with the imaging procedure is automatically selected. Then at block 504 the imaging configuration is loaded or configured so that all parameters are set for performing the imaging procedure. The imaging configuration may include various parameters such as frequency, a speckle reduction imaging, time gain compensation, scan depth, scan format, image frame rate, field of view, focal point, scan lines per image frame, number of ultrasound beams and pitch of the transducer elements. These parameters vary for different imaging configurations. As the imaging configuration is automatically selected and configured in the portable medical imaging device manual interventions of the user is eliminated.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The methods described in conjunction with FIGS. 5, 6 and 7 can be performed using a processor or any other processing device. The method steps can be implemented using coded instructions (e.g., computer readable instructions) stored on a tangible computer readable medium. The tangible computer readable medium may be for example a flash memory, a read-only memory (ROM), a random access memory (RAM), any other computer readable storage medium and any storage media. Although the method of automatically configuring the portable medical imaging device for performing imaging is explained with reference to the flow chart of FIGS. 5, 6 and 7, other methods of implementing the method can be employed. For example, the order of execution of each method steps may be changed, and/or some of the method steps described may be changed, eliminated, divide or combined. Further the method steps may be sequentially or simultaneously executed for presenting health state of a subject in a monitoring system.

This written description uses examples to disclose the invention, including the best mode, and also to enable any person skilled in the art to practice the invention, including making and using any computing system or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An portable medical imaging device comprising:
a processor configured to:
analyze location information associated with the portable medical imaging device,
determine whether the location information is linked to a location of performing an imaging procedure,
select an imaging configuration from a plurality of imaging configurations associated with the imaging procedure to be performed in the location, each imaging configuration of the plurality of imaging configurations is associated with a type of imaging procedure, and
load the imaging configuration to perform the imaging procedure; and
at least one memory communicably coupled to the processor, wherein the at least one memory is configured to store the plurality of imaging configurations.

2. The portable medical imaging device according to claim 1, wherein the processor is further configured to set the portable medical imaging device in a standby mode in response to the portable medical imaging device moving out from the location.

3. The portable medical imaging device according to claim 2, wherein the processor is further configured to instruct the portable medical imaging device to shift to a wakeup mode from the standby mode in response to determining the location information is linked to the location of performing the imaging procedure.

4. The portable medical imaging device according to claim 1, wherein the location information comprises Global Positioning System (GPS) coordinates, and the processor is further configured to determine the GPS coordinates when the portable medical imaging device is in transit.

5. The portable medical imaging device according to claim 4, wherein the processor is further configured to present the GPS coordinates in a map indicating the location to a user.

6. The portable medical imaging device according to claim 1, further comprising a transceiver configured to:
communicate with at least one wireless detector positioned in the location to detect the location information associated with the portable medical imaging device, wherein a wireless detector of the at least one wireless detector operates based on one of a Near Field Communication (NFC) technique and a Bluetooth technique; and
send the location information to the processor.

7. The portable medical imaging device according to claim 1, further comprising a transceiver configured to:
communicate with at least one wireless detector in the location; and
receive an identifier associated with the location from the at least one wireless detector, wherein the identifier indicates the type of imaging procedure performed in the location,
wherein the processor is further configured to process the identifier and identify the imaging configuration associated with the imaging procedure from the plurality of imaging configurations.

8. A medical imaging system comprising:
a portable medical imaging device comprising:
a processor configured to
analyze location information associated with the portable medical imaging device,
determine whether the location information is linked to a location of performing an imaging procedure,
select an imaging configuration from a plurality of imaging configurations associated with the imaging procedure to be performed in the location, each imaging configuration of the plurality of imaging configurations is associated with a type of imaging procedure, and
load the imaging configuration to perform the imaging procedure; and
at least one memory communicably coupled to the processor, wherein the at least one memory is configured to store the plurality of imaging configurations

9. The medical imaging system of claim 8, wherein the processor is further configured to:
set the portable medical imaging device in a standby mode in response to the portable medical imaging device moving out from the location; and
instruct the portable medical imaging device to shift to a wakeup mode from the standby mode in response to determining the location information is linked to the location of performing the imaging procedure.

10. The medical imaging system of claim 8, further comprising:
at least one wireless detector positioned in the location and configured to communicate with the portable medical imaging device, wherein the at least one wireless detector is configured to:
detect a presence of the portable medical imaging device in the location,
transmit the location information to the portable medical imaging device, and
transmit an identifier associated with the location to the portable medical imaging device,
wherein the identifier indicates the type of imaging procedure performed in the location,
wherein the portable medical imaging device further comprises a transceiver configured to receive the identifier, and
wherein the processor is further configured to process the identifier and identify the imaging configuration associated with the imaging procedure from the plurality of imaging configurations.
